(19) United States
(12) Patent Application Publication
(10) Pub. No.: US 2024/0409934 A1
(43) Pub. Date: Dec. 12, 2024

(54) FOXO1-TARGETED THERAPY FOR THE TREATMENT OF CANCER

(71) Applicant: BOARD OF REGENTS, THE UNIVERSITY OF TEXAS SYSTEM, Austin, TX (US)

(72) Inventors: Megan KENIRY, Edinburg, TX (US); David FLORES, Edinburg, TX (US); Alma LOPEZ, Edinburg, TX (US); Shreya UDAWANT, Edinburg, TX (US); Bonnie GUNN, Edinburg, TX (US)

(21) Appl. No.: 18/704,435

(22) PCT Filed: Oct. 25, 2022
(86) PCT No.: PCT/US2022/078628

§ 371 Date: Apr. 24, 2024

Related U.S. Application Data
(60) Provisional application No. 63/271,289, filed on Oct. 25, 2021.

(51) Int. Cl. C12N 15/113; A61K 31/167; A61K 31/337; A61K 31/472; A61K 31/506; A61K 31/517; A61K 31/5377; A61K 31/555; A61K 31/56; A61K 33/243; A61K 45/06; A61P 35/00; C07K 16/30

(52) U.S. Cl. CPC C12N 15/113; A61K 31/167; A61K 31/337; A61K 31/472; A61K 31/506; A61K 31/517; A61K 31/5377; A61K 31/555; A61K 31/56; A61K 33/243; A61K 45/06; A61P 35/00; C07K 16/30; C12N 2310/14

(57) ABSTRACT

The present disclosure provides methods of treating cancer by inhibiting the FOXO1 pathway, such as by small molecular inhibitor or RNA interference. The methods may be used to treat aggressive cancers, such as glioblastoma and basal breast cancer. Further provided herein are combination therapies for the treatment of cancer.

### PRIORITY CLAIM

This application claims the benefit of U.S. Provisional Patent Application No. 63/271,289, filed Oct. 25, 2021, which is incorporated herein by reference in its entirety.

This invention was made with government support under grant number 1SC3GM132053-02 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Oct. 20, 2022, is named UTFRP0035WO.xml and is 9,386 bytes in size.

### BACKGROUND

### 1. Field

The present invention relates generally to the field of molecular biology and medicine. More particularly, it concerns methods of treating cancers, such as breast cancer and brain cancer.

### 2. Description of Related Art

Basal-like breast cancer (BBC) and glioblastoma multiforme (GBM) are aggressive cancers associated with poor prognosis. Weinberg and colleagues first discovered through gene expression profiling that BBC and GBM harbor stem-like gene expression signatures (Ben-Porath et al., 2008). Subsequent work revealed that the Forkhead box transcription factor FOXO1 helped to induce stem gene expression in examined BBC and GBM cell lines (Martinez et al., 2020; Firat et al., 2016). Reduction of FOXO1 and FOXO3 transcription factors led to reduced protein expression of SOX2 and NESTIN in patient derived GBM models (Firat et al., 2016). FOXO1 was also found to direct SOX2 and OCT4 gene expression in glioblastoma cell lines such as U87MG (Martinez et al., 2020). Furthermore, FOXO transcription factors sustain stem cells in an array of contexts including embryonic, hematopoietic, and neural (Zhang et al., 2011; Miyamoto et al., 2007; Kim et al., 2015). However, the full spectrum of contributions that FOXO factors harbor in stem cell contexts remain to be fully delineated.

### SUMMARY

In one embodiment, the present disclosure provides a method for treating or preventing cancer in a patient comprising administering an effective amount of a FOXO1 inhibitor to the patient. In some aspects, the method is preventative for initiation or progression of the cancer including recurrent cancer.

In some aspects, the FOXO1 inhibitor is 5-Amino-7-(cyclohexylamino)-1-ethyl-6-fluoro-4-oxo-1,4-dihydroquinoline-3-carboxylic acid (also referred to herein as AS1842856), 2-cyclopentyl-N-[2,4-dichloro-3-(isoquinolin-5-yloxymethyl) phenyl] N-methylacetamide (also referred to herein as AS1708727), or 3β-Hydroxy-11-oxoolean-12-en-30-oic acid 3-hemisuccinate (also referred to herein as carbenoxolone). In certain aspects, the FOXO1 inhibitor comprises short interfering RNA (siRNA), short hairpin (shRNA). In particular aspects, the FOXO1 inhibitor comprises siRNA. In specific aspects, the siRNA is endoribonuclease-prepared siRNA (esiRNA). In some aspects, the esiRNA comprises a cDNA target sequence of SEQ ID NO: 1.

In certain aspects, the cancer is an aggressive cancer. In some aspects, the cancer is brain cancer, breast cancer, or colon cancer. In particular aspects, the brain cancer is glioblastoma. In specific aspects, the breast cancer is basal breast cancer or triple negative breast cancer. In some aspects, the cancer is recurrent.

In additional aspects, the method further comprises administering an additional anti-cancer therapy. In some aspects, the additional anti-cancer therapy comprises chemotherapy, gene therapy, surgery, hormonal therapy, anti-angiogenic therapy or immunotherapy. In particular aspects, the additional anti-cancer therapy is chemotherapy. In some aspects, the chemotherapy comprises vorinostat, temozolomide, cisplatin, carboplatin, paclitaxel or a combination thereof. In certain aspects, the additional anti-cancer therapy is a receptor tyrosine kinase inhibitor. In some aspects, the receptor tyrosine kinase inhibitor is imatinib. In particular aspects, the receptor tyrosine kinase inhibitor is an EGFR inhibitor. In some aspects, the EGFR inhibitor is erlotinib or gefitinib. In certain aspects, the additional anti-cancer therapy is trastuzumab.

In some aspects, the FOXO1 inhibitor and additional anti-cancer therapy are administered in the same composition. In certain aspects, the FOXO1 inhibitor and additional anti-cancer therapy are administered in separate compositions. In some aspects, the patient has cancer cells with increased FOXO1 expression as compared to a control. In certain aspects, the method comprises administering more than one additional anti-cancer therapy. In some aspects, the patient has been previously administered an anti-cancer therapy. In certain aspects, the anti-cancer therapy is chemotherapy. In some aspects, the patient had low or no response to the chemotherapy. In certain aspects, the method results in increased apoptotic gene expression as compared to expression prior to administering the FOXO1 inhibitor. In some aspects, the increased apoptotic gene expression comprises increased expression of FAS and BIM. In particular aspects, the patient is a human. In specific aspects, the FOXO1 inhibitor and/or additional anti-cancer therapy are administered two or more times.

In another embodiment, there is provided a composition comprising an effective amount of a FOXO1 inhibitor for use in the treatment of a cancer in a patient. In some aspects, the FOXO1 inhibitor is 5-Amino-7-(cyclohexylamino)-1-ethyl-6-fluoro-4-oxo-1,4-dihydroquinoline-3-carboxylic acid, 2-cyclopentyl-N-[2,4-dichloro-3-(isoquinolin-5-yloxymethyl) phenyl] N-methylacetamide, or 3β-Hydroxy-11-oxoolean-12-en-30-oic acid 3-hemisuccinate. In certain aspects, the FOXO1 inhibitor comprises siRNA or shRNA. In particular aspects, the FOXO1 inhibitor comprises siRNA. In specific aspects, the siRNA is esiRNA. In some aspects, the esiRNA comprises a cDNA target sequence of SEQ ID NO: 1.

In additional aspects, the composition further comprises an additional anti-cancer therapy. In some aspects, the additional anti-cancer therapy comprises chemotherapy, gene therapy, surgery, hormonal therapy, anti-angiogenic therapy or immunotherapy. In particular aspects, the additional anti-cancer therapy is chemotherapy. In some aspects, the chemotherapy comprises vorinostat, temozolomide, cisplatin, carboplatin, paclitaxel or a combination thereof. In certain aspects, the additional anti-cancer therapy is a receptor tyrosine kinase inhibitor. In some aspects, the receptor tyrosine kinase inhibitor is imatinib. In particular aspects, the receptor tyrosine kinase inhibitor is an EGFR inhibitor. In some aspects, the EGFR inhibitor is erlotinib or gefitinib. In certain aspects, the additional anti-cancer therapy is trastuzumab.

A further embodiment provides the use of a composition comprising an effective amount of a FOXO1 inhibitor for the treatment of cancer in a patient.

In some aspects, the FOXO1 inhibitor is 5-Amino-7-(cyclohexylamino)-1-ethyl-6-fluoro-4-oxo-1,4-dihydroquinoline-3-carboxylic acid, 2-cyclopentyl-N-[2,4-dichloro-3-(isoquinolin-5-yloxymethyl) phenyl] N-methylacetamide, or 3β-Hydroxy-11-oxoolean-12-en-30-oic acid 3-hemisuccinate. In certain aspects, the FOXO1 inhibitor comprises siRNA or shRNA. In particular aspects, the FOXO1 inhibitor comprises siRNA. In specific aspects, the siRNA is endoribonuclease-prepared siRNA (esiRNA). In some aspects, the esiRNA comprises a cDNA target sequence of SEQ ID NO: 1.

In certain aspects, the use further comprises an additional anti-cancer therapy. In some aspects, the additional anti-cancer therapy comprises chemotherapy, gene therapy, surgery, hormonal therapy, anti-angiogenic therapy or immunotherapy. In particular aspects, the additional anti-cancer therapy is chemotherapy. In some aspects, the chemotherapy comprises vorinostat, temozolomide, cisplatin, carboplatin, paclitaxel or a combination thereof. In certain aspects, the additional anti-cancer therapy is a receptor tyrosine kinase inhibitor. In some aspects, the receptor tyrosine kinase inhibitor is imatinib. In particular aspects, the receptor tyrosine kinase inhibitor is an EGFR inhibitor. In some aspects, the EGFR inhibitor is erlotinib or gefitinib. In certain aspects, the additional anti-cancer therapy is trastuzumab.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1F: FOXO1 Inhibitor Treatment Reduced Colony Formation. Indicated basal breast cancer (MDA-MB-468 (FIG. 1A) or BT549 (FIG. 1B)) or GBM cell lines (LN229 (FIG. 1C), DBTRG (FIG. 1D), A172 (FIG. 1E) or LN18 (FIG. 1F)) were treated with FOXO1 inhibitor AS1842856 for five days and subsequently stained with crystal violet. The FOXO1 inhibitor decreased colony formation in these cell lines. * denotes significantly different by Tukey Test compared to indicated samples (p<0.05).

FIGS. 2A-2F: FOXO1 Inhibitor Treatment led to the induction of pro-apoptotic genes. Indicated cell lines (BT549 (FIG. 2A), MDA-MB-468 (FIG. 2B), DBTRG (FIG. 2C), A172 (FIG. 2D), LN229 (FIG. 2E), and LN18 (FIG. 2F)) were treated with 1 μM AS1842856 for 48 hours and examined for changes in gene expression by qRT-PCR using TUBB as the reference gene. It was found that FOXO1 inhibitor treatment induced apoptotic genes FAS and/or BIM in these cell lines. * denotes significantly different by Tukey Test compared to the control (p<0.05).

FIGS. 3A-3D: FOXO1 Inhibitor Treatment led to Apoptosis. (FIGS. 3A-3C) LN229 and BT549 cells were treated with 1 μM AS1842856 and were stained with propidium iodide and FITC-Annexin V followed by flow cytometric analyses. Apoptosis increased in LN229 and BT549 cells after FOXO1 inhibitor treatment as measure by PI and/or Annexin V staining. * denotes significantly different by Tukey Test than the control (P<0.05). (FIG. 3D) Western blot analyses were performed with BT549 and LN229 samples that were treated with AS1842856. Both cell lines had increased caspase 3 cleavage.

FIGS. 4A-4F: Inhibition of FOXO1 reduced colony number. (FIG. 4A) U87MG cells were treated with AS1842856; colony number was not significantly impacted. (FIGS. 4B-4C) Indicated colon cancer cells (HCT116 or SW480) were treated with FOXO1 inhibitor AS1842856 for five days and subsequently stained with crystal violet. FOXO1 inhibition decreased colony formation in these cell lines. (FIGS. 4D-4F) Treatment with another FOXO1 inhibitor AS1708727 in BT549, MDA-MB-468 and LN18 also led to reduced colony formation. * denotes significantly different by Tukey Test compared to indicated sample (p<0.05).

FIGS. 5A-5G: FOXO1 Inhibitor Treatment led to the induction of pro-apoptotic genes. (FIGS. 5A-5B) Indicated cell lines were treated with 1 μM AS1842856 for 48 hours and examined for changes in gene expression by qRT-PCR using TUBB as the reference gene. It was found that FOXO1 inhibitor treatment induced apoptotic genes FAS and/or BIM in U87MG and HCT116 cell lines. (FIGS. 5C-5D) Treatment with AS1708727 led to FAS induction in BT549 (1 μM treatment for 4 days) and MDA-MB-468 cells (1 μM treatment for two days). (FIGS. 5E-5G) FOXO1 expression was assessed in indicated cell line. * denotes significantly different by Tukey Test compared to control (p<0.05).

FIGS. 6A-6D: FOXO1 RNAi treatment led to the induction of FAS in MDA-MB-468 cells. (FIGS. 6A-6C) Indicated cell lines were treated with FOXO1 esiRNA or control for 72 hours and examined for changes in gene expression by qRT-PCR using TUBB as the reference gene. It was found that FOXO1 esiRNA treatment-induced FAS in MDA-MB-468 cells. FOXO1 RNAi was not sustained in BT549 cells. (FIG. 6D) 5AZA treatment (3 μM for 48 hours) led to induction of FAS, and BIM in BT549 cells * denotes significantly different by Tukey Test compared to the control (P<0.05).

FIGS. 7A-7B: Impacts of AS1842856 treatment on FOXO1 gene expression was cell type dependent. (FIGS. 7A-7B) FOX1 was induced by AS1842856 in BT549 cells, but not in MDA-MB-468 cells. * denotes significantly different by Tukey Test compared to control (P<0.05).

FIGS. 8A-8B: AS1842856 treatment led to the induction of pro-apoptotic genes in cancer stem cells. (FIGS. 8A-8B) Cancer stem cells (CSCs) from indicated cell lines were treated with 1 μM AS1842856 for 48 hours and examined for changes in gene expression by qRT-PCR using TUBB as the reference gene. It was found that AS1842856 treatment induced apoptotic genes FAS and/or BIM in BT549 CSCs and U87MG CSCs. * denotes significantly different by Tukey Test compared to control (P<0.05).

### DESCRIPTION OF ILLUSTRATIVE EMBODIMENTS

Basal-like breast cancer has characteristics in common with myoepithelial cells of the breast and are typically triple negative (lack expression of the estrogen receptor (ER), progesterone receptor (PR) and HER2 receptor) (Bertucci et al., 2012). This breast cancer is commonly found in younger and African American women. BBC is specifically associated with poor prognosis and chemotherapeutic resistance. In terms of therapeutics BBC are frequently triple negative and therefore respond currently only to conventional chemotherapy (Diana et al., 2020).

GBM is an aggressive brain cancer that has a five-year survival rate of 6.8% (Anjum et al., 2017). GBM patients on average have a survival length between 12 and 18 months (Anjum et al., 2017). Of all malignant brain tumors GBM is the most common type found in adults.

BBC and GBM are poor prognosis cancers that lack effective targeted therapies. The present studies examined the impact of FOXO1 inhibition on BBC and GBM cell viability. A set of cell lines were treated with increasing concentrations of FOXO1 inhibitor; it was found that FOXO1 inhibition induced apoptosis in the BBC and GBM cell lines. Treatment of BBC and GBM cancer cell lines with FOXO1 inhibitor led to increased FAS and BIM gene expression as well as positive Annexin V and propidium iodide staining. Thus, targeting BBC and GBM with FOXO1 inhibitor treatment can expand the repertoire of therapies for these poor prognosis cancers.

Accordingly, in certain embodiments, the present disclosure provides methods for the treatment of cancers, such as BBC and GBM, by inhibiting FOXO1, such as by administration of AS1842856. Further provided here are combination therapies for the treatment of aggressive cancers.

### I. Methods of Treatment

Further provided herein are methods for treating or delaying progression of cancer in an individual comprising administering a FOXO1 inhibitor alone or in combination with an additional therapy.

Examples of cancers contemplated for treatment include lung cancer, head and neck cancer, breast cancer, brain cancer, pancreatic cancer, prostate cancer, renal cancer, bone cancer, testicular cancer, cervical cancer, gastrointestinal cancer, lymphomas, pre-neoplastic lesions in the lung, colon cancer, melanoma, and bladder cancer. In particular aspects, the cancer is basal breast cancer or glioblastoma.

The cancer may specifically be of the following histological type, though it is not limited to these: neoplasm, malignant; carcinoma; non-small cell lung cancer; renal cancer; renal cell carcinoma; clear cell renal cell carcinoma; lymphoma; blastoma; sarcoma; carcinoma, undifferentiated; meningioma; brain cancer; oropharyngeal cancer; nasopharyngeal cancer; biliary cancer; pheochromocytoma; pancreatic islet cell cancer; Li-Fraumeni tumor; thyroid cancer; parathyroid cancer; pituitary tumor; adrenal gland tumor; osteogenic sarcoma tumor; neuroendocrine tumor; breast cancer; lung cancer; head and neck cancer; prostate cancer; esophageal cancer; tracheal cancer; liver cancer; bladder cancer; stomach cancer; pancreatic cancer; ovarian cancer; uterine cancer; cervical cancer; testicular cancer; colon cancer; rectal cancer; skin cancer; giant and spindle cell carcinoma; small cell carcinoma; small cell lung cancer; papillary carcinoma; oral cancer; oropharyngeal cancer; nasopharyngeal cancer; respiratory cancer; urogenital cancer; squamous cell carcinoma; lymphoepithelial carcinoma; basal cell carcinoma; pilomatrix carcinoma; transitional cell carcinoma; papillary transitional cell carcinoma; adenocarcinoma; gastrointestinal cancer; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma with squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; lentigo maligna melanoma; acral lentiginous melanoma; nodular melanoma; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; an endocrine or neuroendocrine cancer or hematopoietic cancer; pinealoma, malignant; chordoma; central or peripheral nervous system tissue cancer; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; B-cell lymphoma; malignant lymphoma; Hodgkin's disease; Hodgkin's; low grade/follicular non-Hodgkin's lymphoma; paragranuloma; malignant lymphoma, small lymphocytic; malignant lymphoma, large cell, diffuse; malignant lymphoma, follicular; mycosis fungoides; mantle cell lymphoma; Waldenstrom's macroglobulinemia; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; chronic lymphocytic leukemia (CLL); acute lymphoblastic leukemia (ALL); Hairy cell leukemia; chronic myeloblastic leukemia; and/or hairy cell leukemia.

In some embodiments, the subject is a mammal, e.g., a primate, preferably a higher primate, e.g., a human (e.g., a patient having, or at risk of having, a disorder described herein). In one embodiment, the subject is in need of enhancing an immune response. In certain embodiments, the subject is, or is at risk of being, immunocompromised. For example, the subject is undergoing or has undergone a chemotherapeutic treatment and/or radiation therapy. Alternatively, or in combination, the subject is, or is at risk of being, immunocompromised as a result of an infection.

### A. FOXO1 Inhibition

Conserved FOXO-1, -3 and -4 transcription factors are partially redundant and negatively regulated by AKT (Paik et al., 2009; Paik et al., 2007). These factors act in a context-dependent manner to regulate metabolism by activating gluconeogenesis and impacting mitochondrial function (Calnan and Brunet, 2008; Carter and Brunet, 2007; Matsumoto and Accili, 2005). The best described role for FOXO factors in cancer is to serve as tumor suppressors that induce genes such as TRAIL to promote apoptosis and p27 to halt the cell cycle (Calnan and Brunet, 2008; Carter and Brunet, 2007). Emerging evidence points to pro-oncogenic roles for some FOXO factors in a set of cancers such as Diffuse Large B Cell

Lymphoma (DLBCL) in which FOXO1 is commonly mutated to a constitutively nuclear form; these FOXO1 mutations were associated with poor prognosis (Trinh et al., 2013).

The phosphatidylinositol 3 kinase (PI3K) pathway promotes cell growth, proliferation, and migration in BBC and GBM cells (Lu et al., 2003; Lucas et al., 2010). Receptor tyrosine kinases (RTKs) such as epidermal growth factor receptor (EGFR) are bound by ligand, leading to dimerization and auto-phosphorylation (Chakravarti et al., 2004). This creates docking sites on the RTKs that among other things activate the lipid kinase PI3K, which phosphorylates phosphatidylinositol 4,5 bisphosphate (PIP2) on the D3 position to produce phosphatidylinositol 3,4,5 tris phosphate (PIP3) (Lu et al., 2003; Lucas et al., 2010). Lipid second messenger PIP3 binds to and activates targets such as AKT to promote growth and survival. AKT has over twenty identified targets including FOXO-1, -3 and -4 transcription factors on conserved residues, typically leading to their cytoplasmic sequestration/inactivation (Brunet et al., 1999; Brunet et al., 2002). However, a subset of FOXO transcription factors reside in the nucleus via unknown mechanisms in BBC and GBM despite constitutively active PI3K pathway activity (Keniry et al., 2013).

Epigenetics and mutations lead to nearly uniform constitutively active PI3K pathway activity in BBC and GBM (Saal et al., 2008; Saal et al., 2005; Saal et al., 2007). Commonly the dual specificity phosphatase PTEN (which acts as a lipid phosphatase to diminish cellular pools of PIP3) is mutated to an inactive form in BBC and GBM (Saal et al., 2008; Saal et al., 2005). EGFR is frequently mutated to a constitutively active form in these cancers (Pires et al., 2013). These changes greatly contribute to cancer formation, progression and therapeutic resistance.

A FOXO1 inhibitor may induce a transition from quiescence GO to the G1 phase of the cell cycle, by this reversing HIV-1 latency in T lymphocytes. The use of the inhibitor of the can induce both bioenergetics and transcriptional activities of T cells, together with a significant increase of their cell size, but without any cell division. The FOXO1 inhibitor can allow SAMHD1 phosphorylation. SAMHD1 is a cellular quiescence factor and a well-known restriction factor of HIV infection. This phosphorylation correlates with loss of its ability to restrict HIV.

In some aspects, the inhibitor may be a low molecular weight compound, e.g. a small organic molecule. In some aspects, small organic molecules range in size up to about 10000 Da, more particularly up to 5000 Da, more particularly up to 2000 Da and most particularly up to about 1000 Da.

In some aspects, the present methods and compositions comprise FOXO1 inhibition, such as with a compound of the formula (also referred to herein as AS1842856 (Nagashima et al., 2010)):

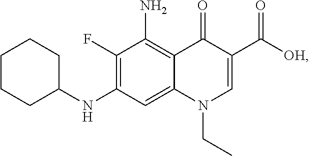 [SMILES: CCn1cc(C(=O)O)c(=O)c2c(N)c(F)c(NC3CCCCC3)cc21]

or a pharmaceutically acceptable salt thereof. In one aspect, the compound is 5-Amino-7-(cyclohexylamino)-1-ethyl-6-fluoro-4-oxo-1,4-dihydroquinoline-3-carboxylic acid.

In some aspects, the present methods and compositions comprise FOXO1 inhibition, such as with a compound of the formula (also referred to herein as AS1708727 (Tanaka et al., 2010)):

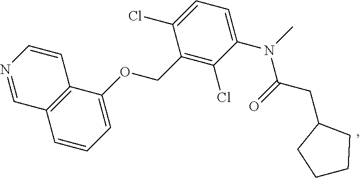 [SMILES: CN(C(=O)CC1CCCC1)c1ccc(Cl)c(COc2cccc3cnccc23)c1Cl]

or a pharmaceutically acceptable salt thereof. In one aspect, the compound is 2-cyclopentyl-N-[2,4-dichloro-3-(isoquinolin-5-yloxymethyl) phenyl] N-methylacetamide.

In some aspects, the present methods and compositions comprise FOXO1 inhibition with a compound of the formula (also referred to herein as carbenoxolone and enoxolone succinate (Salcher et al., 2020)):

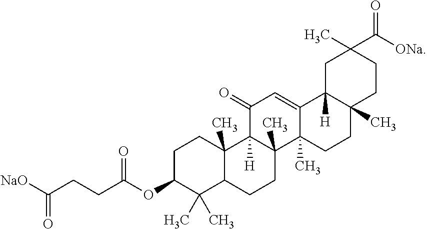

or a pharmaceutically acceptable salt thereof. In one aspect, the compound is 3β-Hydroxy-11-oxoolean-12-en-30-oic acid 3-hemisuccinate. In certain aspects, the FOXO1 inhibitor is carbenoxolone (Salcher et al., 2020; incorporated herein by reference).

FOXO1 may be inhibited or disrupted by RNAi, such as siRNA or shRNA, or by sequence-specific or targeted nucleases. In some aspects, the FOXO1 inhibitor is a compound (e.g., compounds 1-13 of the formulas shown below) disclosed in Langlet et al., 2017; incorporated herein by reference.

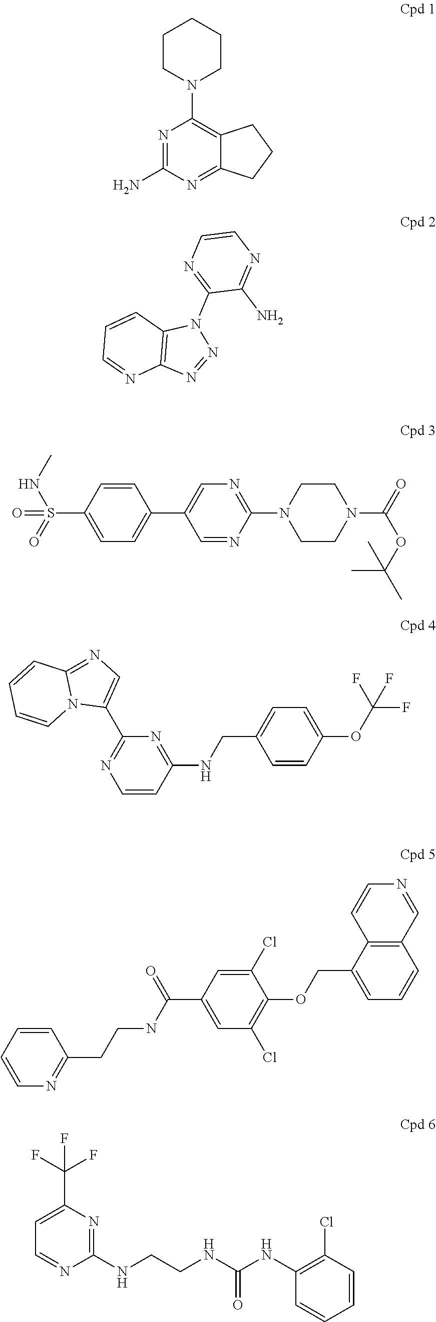 [SMILES: Nc1nc2c(c(N3CCCCC3)n1)CCC2 ; Nc1nccnc1-n1nnc2ncccc21 ; CNS(=O)(=O)c1ccc(-c2cnc(N3CCN(C(=O)OC(C)(C)C)CC3)nc2)cc1 ; FC(F)(F)Oc1ccc(CNc2ccnc(-c3cnc4ccccn34)n2)cc1 ; O=C(NCCc1ccccn1)c1cc(Cl)c(OCc2cccc3cnccc23)c(Cl)c1 ; O=C(NCCNc1nccc(C(F)(F)F)n1)Nc1ccccc1Cl]

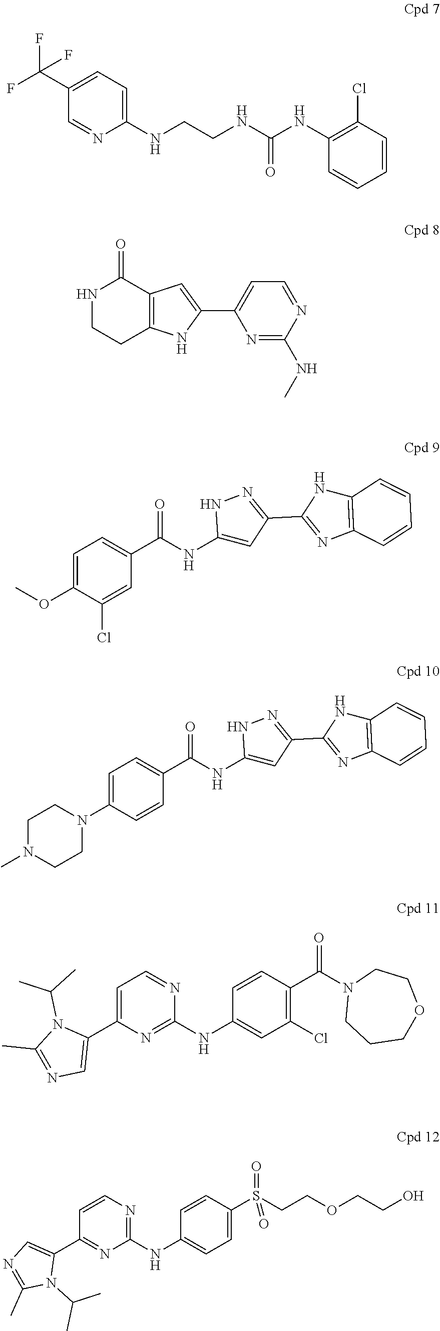 [SMILES: O=C(NCCNc1ccc(C(F)(F)F)cn1)Nc1ccccc1Cl ; CNc1nccc(-c2cc3c([nH]2)CCNC3=O)n1 ; COc1ccc(C(=O)Nc2cc(-c3nc4ccccc4[nH]3)n[nH]2)cc1Cl ; CN1CCN(c2ccc(C(=O)Nc3cc(-c4nc5ccccc5[nH]4)n[nH]3)cc2)CC1 ; Cc1ncc(-c2ccnc(Nc3ccc(C(=O)N4CCCOCC4)c(Cl)c3)n2)n1C(C)C ; Cc1ncc(-c2ccnc(Nc3ccc(S(=O)(=O)CCOCCO)cc3)n2)n1C(C)C]

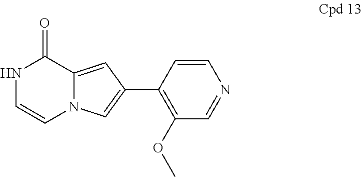 [SMILES: COc1cnccc1-c1cc2c(=O)[nH]ccn2c1]

As used herein, a “disruption” of a gene refers to the elimination or reduction of expression of one or more gene products encoded by the subject gene in a cell, compared to the level of expression of the gene product in the absence of the disruption. Exemplary gene products include mRNA and protein products encoded by the gene. Disruption in some cases is transient or reversible and in other cases is permanent. Disruption in some cases is of a functional or full-length protein or mRNA, despite the fact that a truncated or non-functional product may be produced. In some embodiments herein, gene activity or function, as opposed to expression, is disrupted. Gene disruption is generally induced by artificial methods, i.e., by addition or introduction of a compound, molecule, complex, or composition, and/or by disruption of nucleic acid of or associated with the gene, such as at the DNA level. Exemplary methods for gene disruption include gene silencing, knockdown, knockout, and/or gene disruption techniques, such as gene editing. Examples include antisense technology, such as RNAi, siRNA, shRNA, and/or ribozymes, which generally result in transient reduction of expression, as well as gene editing techniques which result in targeted gene inactivation or disruption, e.g., by induction of breaks and/or homologous recombination. Examples include insertions, mutations, and deletions. The disruptions typically result in the repression and/or complete absence of expression of a normal or “wild type” product encoded by the gene. Exemplary of such gene disruptions are insertions, frameshift and missense mutations, deletions, knock-in, and knock-out of the gene or part of the gene, including deletions of the entire gene. Such disruptions can occur in the coding region, e.g., in one or more exons, resulting in the inability to produce a full-length product, functional product, or any product, such as by insertion of a stop codon. Such disruptions may also occur by disruptions in the promoter or enhancer or other region affecting activation of transcription, so as to prevent transcription of the gene. Gene disruptions include gene targeting, including targeted gene inactivation by homologous recombination.

For example, the disruption can be affected be sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, gene disruption is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

In some embodiments, the disruption is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease. Zinc finger, TALE, and CRISPR system binding domains can be engineered to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 2011/0301073.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone nonhomologous end joining (NHEJ) and homology-directed repair (HDR). In some embodiments the nuclease is an endonuclease, such as a zinc finger nuclease (ZFN), TALE nuclease (TALEN), and RNA-guided endonuclease (RGEN), such as a CRISPR-associated (Cas) protein, or a meganuclease.

The term “siRNA” (short interfering RNA) refers to short double stranded RNA complex, typically 19-28 base pairs in length. In other words, siRNA is a double-stranded nucleic acid molecule comprising two nucleotide strands, each strand having about 19 to about 28 nucleotides (i.e., about 19, 20, 21, 22, 23, 24, 25, 26, 27, or 28 nucleotides). The complex often includes a 3′-overhang. siRNA can be made using techniques known to one skilled in the art and a wide variety of siRNA is commercially available from suppliers such as Integrated DNA Technologies, Inc. (Coralville, Iowa).

The size of the RNAi loaded used herein may be less than 100 nucleotides in length, such as less than 75 nucleotides, particularly less than 50 nucleotides in length. For example, the RNA may have a length of about 10-100 nucleotides, such as 20-50 nucleotides, particularly 10-20, 15-25, 20-30, 25-35, 30-40, or 45-50 nucleotides.

The RNAi may be modified or non-modified. The RNAi may comprise an alteration of one or more nucleotides. Such alterations can include the addition of non-nucleotide material, such as to the end(s) of the RNAi or internally (at one or more nucleotides of the RNA). In certain aspects, the RNAi molecule contains a 3′-hydroxyl group. Nucleotides in the RNAi molecules of the present disclosure can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation). Collectively, all such altered nucleic acids or RNAs described above are referred to as modified siRNAs. The RNAi may be conjugated or encapsulated for delivery, such as to lipids or nanoparticles.

Preferably, RNAi is capable of decreasing the expression of a protein by at least 10%, 20%, 30%, or 40%, more preferably by at least 50%, 60%, or 70%, and even more preferably by at least 75%, 80%, 90%, 95% or more.

The siRNA as used in the methods or compositions described herein may comprise a portion which is complementary to an mRNA sequence encoded by NCBI Reference Sequence for PTPN1. In an embodiment, the siRNA comprises a double-stranded portion (duplex). In an embodiment, the siRNA is 20-25 nucleotides in length. In an embodiment the siRNA comprises a 19-21 core RNA duplex with a one or 2 nucleotide 3′ overhang on, independently, either one or both strands. In an embodiment, the overhang is UU. The siRNA can be 5′ phosphorylated or not and may be modified with any of the known modifications in the art to improve efficacy and/or resistance to nuclease degradation. In a non-limiting embodiment, the siRNA can be administered such that it is transfected into one or more cells. In one embodiment, a siRNA may comprise a double-stranded RNA comprising a first and second strand, wherein one strand of the RNA is 80, 85, 90, 95 or 100% complementary to a portion of an RNA transcript of a gene.

In one embodiment, a single strand component of a siRNA of the present disclosure is from 14 to 50 nucleotides in length. In another embodiment, a single strand component of a siRNA is 14, 15, 16, 17, 18, 19, 20, 21, 22, 23, 24, 25, 26, 27, or 28 nucleotides in length. In yet another embodiment, a single strand component of a siRNA of the present disclosure is 21 nucleotides in length. In yet another embodiment, a single strand component of a siRNA of the present disclosure is 22 nucleotides in length. In yet another embodiment, a single strand component of a siRNA of the present disclosure is 23 nucleotides in length. In one embodiment, a siRNA of the present disclosure is from 28 to 56 nucleotides in length.

In some aspects, FOXO1 may be inhibited by an anti-FOXO1 antibody or fragment thereof. As used herein, the term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class, including any of the human classes: IgG, IgM, IgA, IgD, and IgE. The antibody may be a bi-specific antibody. In exemplary embodiments, antibodies used with the methods and compositions described herein are derivatives of the IgG class. The term antibody also refers to antigen-binding antibody fragments. Examples of such antibody fragments include, but are not limited to, Fab, Fabÿ, F(abÿ)2, scFv, Fv, dsFv diabody, and Fd fragments. Antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about amino acids and more typically will comprise at least about 200 amino acids.

### B. Pharmaceutical Formulations and Routes of Administration

Where clinical applications are contemplated, it will be necessary to prepare pharmaceutical compositions in a form appropriate for the intended application. In some embodiments, such formulation with the FOXO1 inhibitors of the present disclosure is contemplated. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector to cells, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present disclosure, its use in therapeutic compositions is contemplated. Supplementary active ingredients also can be incorporated into the compositions.

The active compositions of the present disclosure may include classic pharmaceutical preparations. Administration of these compositions according to the present disclosure will be via any common route so long as the target tissue is available via that route. Such routes include oral, nasal, buccal, rectal, vaginal or topical route. Alternatively, administration may be by orthotopic, intradermal, subcutaneous, intramuscular, intratumoral, intraperitoneal, or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the inhibitors described herein may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences,” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA's Division of Biological Standards and Quality Control of the Office of Compliance and Biologics Quality.

In particular, the compositions that may be used in treating a disease or disorder, such as cancer, in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., causing apoptosis of cancerous cells or killing microbes). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms of the infection or cancer and other drugs being administered concurrently. A composition as described herein is typically administered at a dosage that induces death of cancerous cells (e.g., induces apoptosis of a cancer cell), as assayed by identifying a reduction in hematological parameters (complete blood count—CBC), or cancer cell growth or proliferation. In some embodiments, amounts of the ADCs used to induce apoptosis of the cancer cells is calculated to be from about 0.01 mg to about 10,000 mg/day. In some embodiments, the amount is from about 1 mg to about 1,000 mg/day. In some embodiments, these dosings may be reduced or increased based upon the biological factors of a particular patient such as increased or decreased metabolic breakdown of the drug or decreased uptake by the digestive tract if administered orally. Additionally, the ADCs may be more efficacious and thus a smaller dose is required to achieve a similar effect. Such a dose is typically administered once a day for a few weeks or until sufficient reducing in cancer cells has been achieved.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, marker (as defined herein), family history, and the like).

In some embodiments, the disclosure provides a method of monitoring treatment progress. The method includes the step of determining a level of changes in hematological parameters and/or cancer stem cell (CSC) analysis with cell surface proteins as diagnostic markers (which can include, for example, but are not limited to CD34, CD38, CD90, and CD117) or diagnostic measurement (e.g., screen, assay) in a subject suffering from or susceptible to a disorder or symptoms thereof associated with cancer (e.g., leukemia) in which the subject has been administered a therapeutic amount of a composition as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy. In certain preferred embodiments, a pre-treatment level of marker in the subject is determined prior to beginning treatment according to the methods described herein; this pre-treatment level of marker can then be compared to the level of marker in the subject after the treatment commences, to determine the efficacy of the treatment.

For oral administration the oligonucleotides of the present disclosure may be incorporated with excipients. The compositions of the present disclosure may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The RNAi of the present disclosure may be administered by the use of lipid delivery vehicles. Lipid vehicles encompass micelles, microemulsions, macroemulsions, liposomes, and similar carriers. The term micelle refers to colloidal aggregates of amphipathic (surfactant) molecules that are formed at a well-defined concentration known as the critical micelle concentration. Micelles are oriented with the nonpolar portions at the interior and the polar portions at the exterior surface, exposed to water. The typical number of aggregated molecules in a micelle (aggregation number) is 50 to 100. Microemulsions are essentially swollen micelles, although not all micellar solutions can be swollen to form microemulsions. Microemulsions are thermodynamically stable, are formed spontaneously, and contain particles that are extremely small. Droplet diameters in microemulsions typically range from 10-100 nm. In contrast, the term macroemulsions refers to droplets with diameters greater than 100 nm. Liposomes are closed lipid vesicles comprising lipid bilayers that encircle aqueous interiors. Liposomes typically have diameters of 25 nm to 1 μm.

### C. Combination Therapies

It is envisioned that the inhibitors described herein may be used in combination therapies with one or more therapies or a compound which mitigates one or more of the side effects experienced by the patient. It is common in the field of medical therapy to combine therapeutic modalities. The following is a general discussion of therapies that may be used in conjunction with the therapies of the present disclosure.

To treat certain diseases or disorders using the methods and compositions of the present disclosure, one would generally contact the subject with a compound and at least one other therapy. These therapies would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the drug conjugates described herein may precede or follow the other treatment by intervals ranging from minutes to weeks. One would generally ensure that a significant period of time did not expire between the time of each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 1-2 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where a FOXO1 inhibitor is “A,” and the other therapy is “B,” as exemplified below:
A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### 1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid: 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman, gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

### 2. Radiotherapy

Other factors that cause DNA damage and have been used extensively include what are commonly known as y-rays, X-rays, and/or the directed delivery of radioisotopes to tumor cells. Other forms of DNA damaging factors are also contemplated, such as microwaves, proton beam irradiation, and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

### 3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment. As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons a, B, and y, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints are molecules in the immune system that either turn up a signal (e.g., co-stimulatory molecules) or turn down a signal. Inhibitory checkpoint molecules that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12 (4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present invention. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95 (17): 10067-10071; Camacho et al. (2004) J Clin Oncology 22 (145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WOO 1/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesions such as described in U.S. Pat. No. 8,329,867, incorporated herein by reference.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### II. Definitions

As used herein the specification, “a” or “an” may mean one or more. As used herein in the claim(s), when used in conjunction with the word “comprising,” the words “a” or “an” may mean one or more than one.

The term “about” means, in general, within a standard deviation of the stated value as determined using a standard analytical technique for measuring the stated value. The terms can also be used by referring to plus or minus 5% of the stated value.

“Aggressive cancers” as referred to herein are cancers that grow and spread more aggressively and have challenges that make them more difficult to treat than common tumor types. Cancer cells can often become resistant to standard treatment options, and patients may therefore exhaust these options very quickly.

An “anti-cancer” agent is capable of negatively affecting a cancer cell/tumor in a subject, for example, by promoting killing of cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer.

The term “effective,” as that term is used in the specification and/or claims, means adequate to accomplish a desired, expected, or intended result. “Effective amount,” “therapeutically effective amount” or “pharmaceutically effective amount” when used in the context of treating a patient or subject with a compound means that amount of the compound which, when administered to a subject or patient for treating or preventing a disease, is an amount sufficient to effect such treatment or prevention of the disease.

The term “essentially” is to be understood that methods or compositions include only the specified steps or materials and those that do not materially affect the basic and novel characteristics of those methods and compositions.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds of the present invention which are pharmaceutically acceptable, as defined above, and which possess the desired pharmacological activity. Non-limiting examples of such salts include acid addition salts formed with inorganic acids such as hydrochloric acid, hydrobromic acid, sulfuric acid, nitric acid, and phosphoric acid; or with organic acids such as 1,2-ethanedisulfonic acid, 2-hydroxyethanesulfonic acid, 2-naphthalenesulfonic acid, 3-phenylpropionic acid, 4,4′-methylenebis (3-hydroxy-2-ene-1-carboxylic acid), 4-methylbicyclo[2.2.2] oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl) benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, and trimethylacetic acid. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Non-limiting examples of acceptable organic bases include ethanolamine, diethanolamine, triethanolamine, tromethamine, and N-methylglucamine. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prophylactically treating” includes: (1) reducing or mitigating the risk of developing the disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The terms “substantially” or “approximately” as used herein may be applied to modify any quantitative comparison, value, measurement, or other representation that could permissibly vary without resulting in a change in the basic function to which it is related.

As used herein, a composition or media that is “substantially free” of a specified substance or material contains≤30%, ≤20%, ≤15%, more preferably ≤10%, even more preferably <5%, or most preferably ≤1% of the substance or material.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease.

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all the compounds of the present invention are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

### III. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—FOXO Transcription Factors Promote Stem Signature in Aggressive U87MG Cells

### Inhibition of FOXO1 Reduced BBC and GBM Colony Formation.

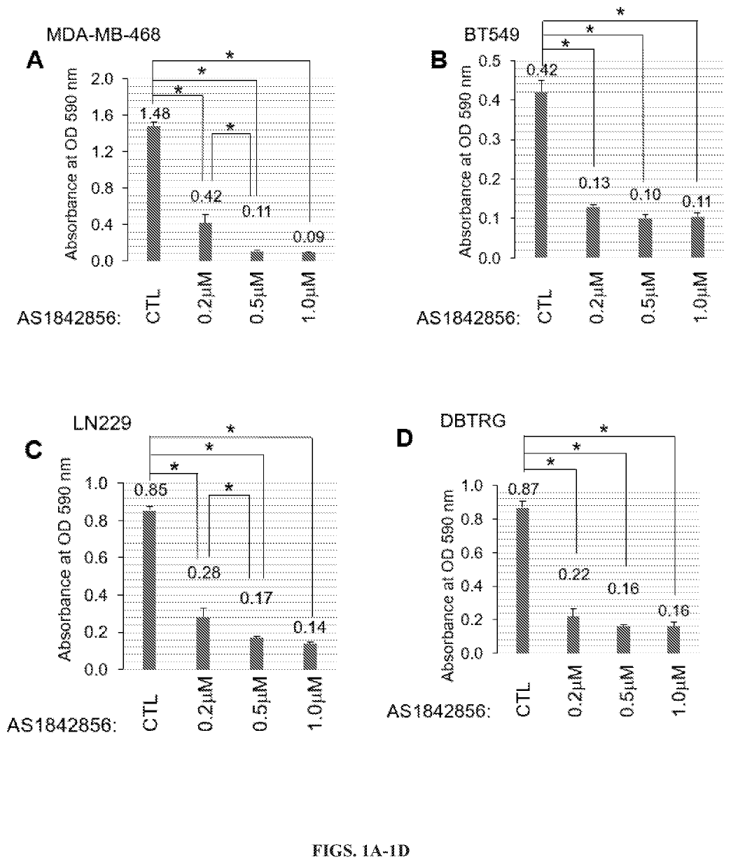

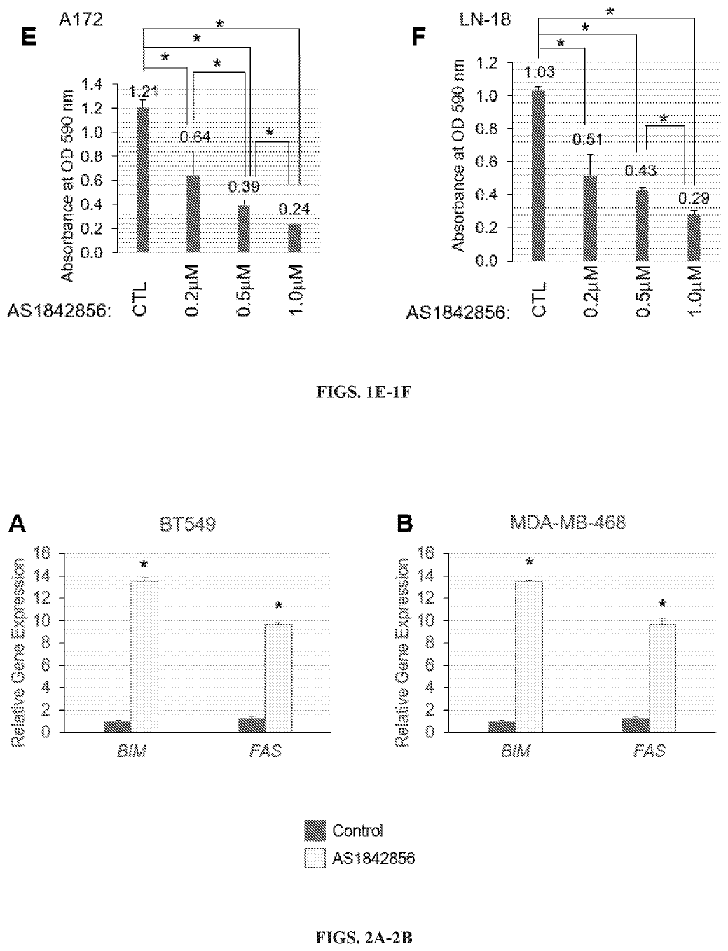

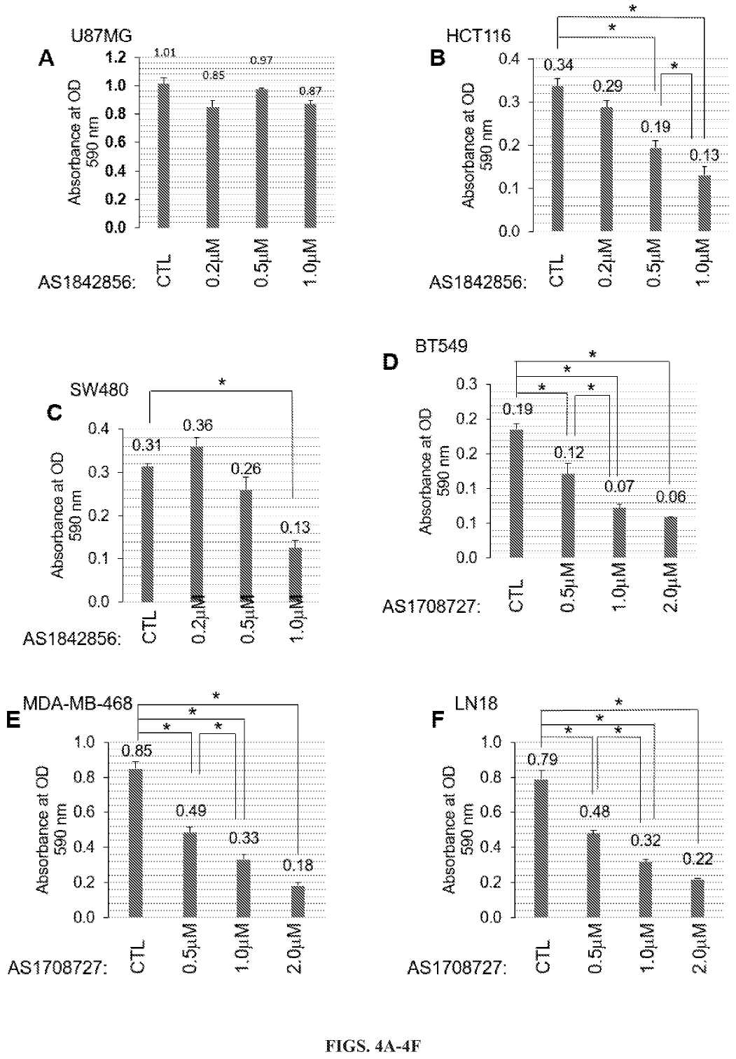

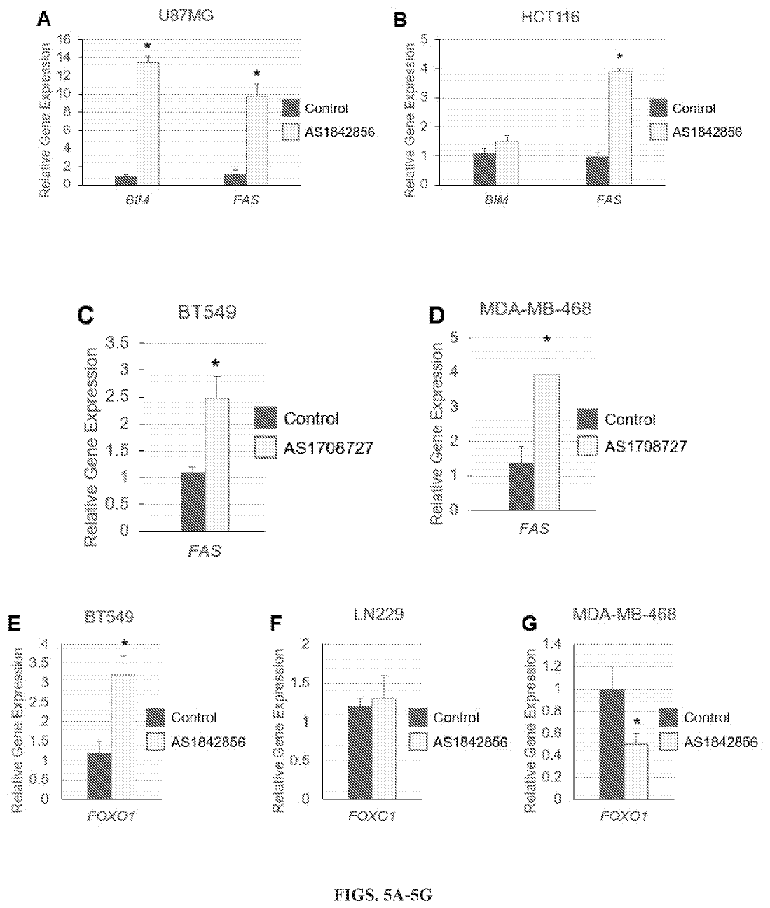

AS1842856 is a selective FOXO1 inhibitor that reduces DNA binding and transactivation (Nagashima et al., 2010). The impact of FOXO1 inhibition on colony formation was examined in a set of cancer cell lines. BBC (MDA-MB-468 and BT549) and GBM (LN229, DBTRG, A172, LN-18) cell lines were treated with increasing amounts of drug 200 nM, 500 nM, and 1.0 μM for five days and stained cells with crystal violet. It was found that all cell lines had fewer cells with 200 nM drug treatment with further reductions using 500 nM and 1 μM drug (FIGS. 1A-1F). U87MG growth was resistant to selective FOXO1 inhibitor treatment. To examine whether FOXO1 inhibition impacted other types of cancer, two colon cancer cell lines were also examined for colony formation inhibition upon selective FOXO1 inhibitor treatment. Significantly reduced colony formation with selective FOXO1 inhibitor was found in HCT116 and SW480 cell lines (FIGS. 4A-4B). Therefore, eight out of nine cell lines examined had reduced colony formation with FOXO1 inhibition using AS1842856. Another FOXO1 inhibitor AS1708727 was also examined in several cell lines for the ability to impact colony formation. AS1708727 treatment reduced colony formation in BT549, MDA-MB-468 and LN18 cell lines (FIGS. 4C-4E) (Tanaka et al., 2010).

### Inhibition of FOXO 1 Induced Pro-Apoptotic Genes in BBC and GBM Cell Lines.

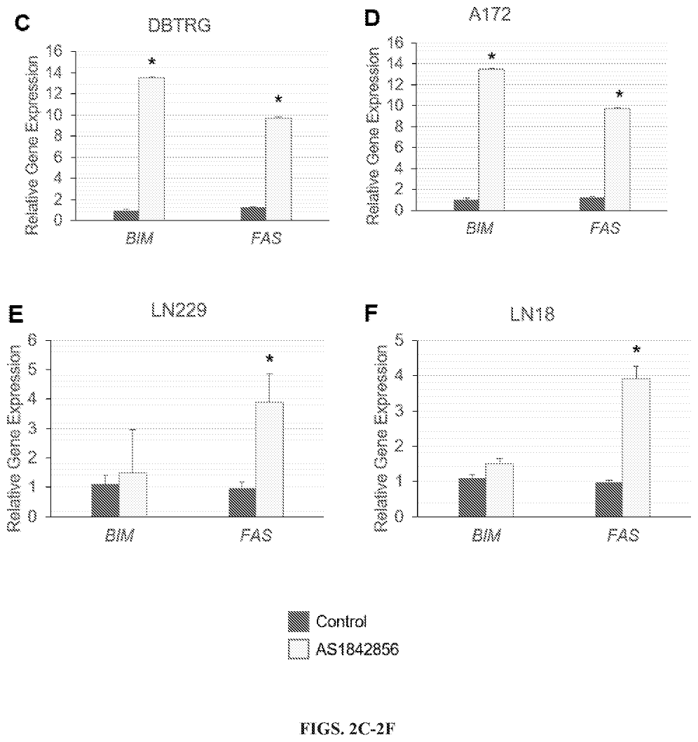

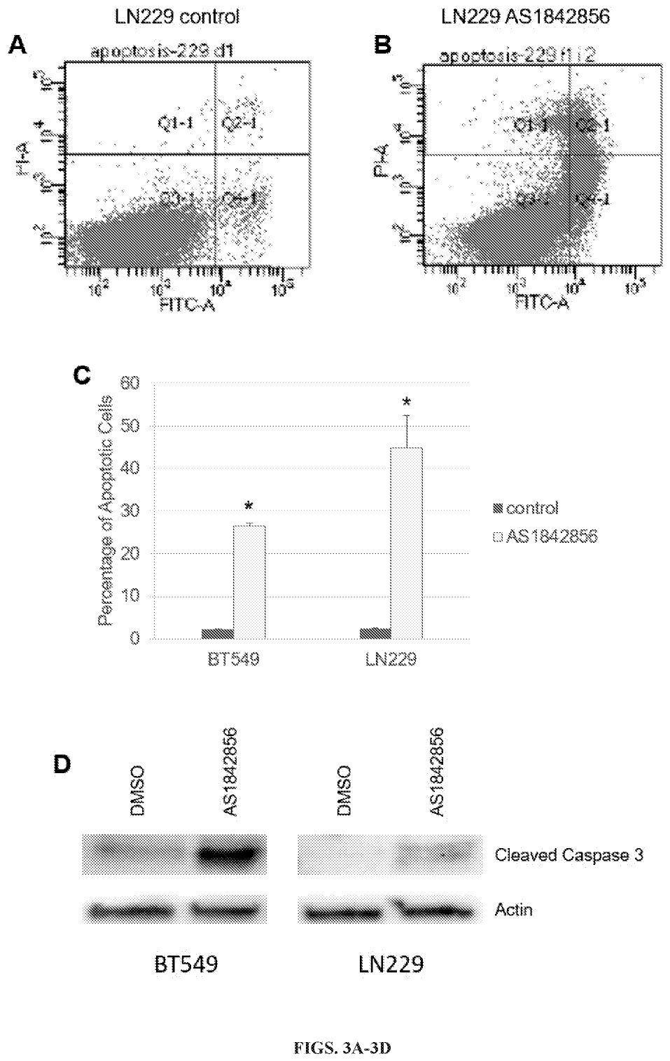

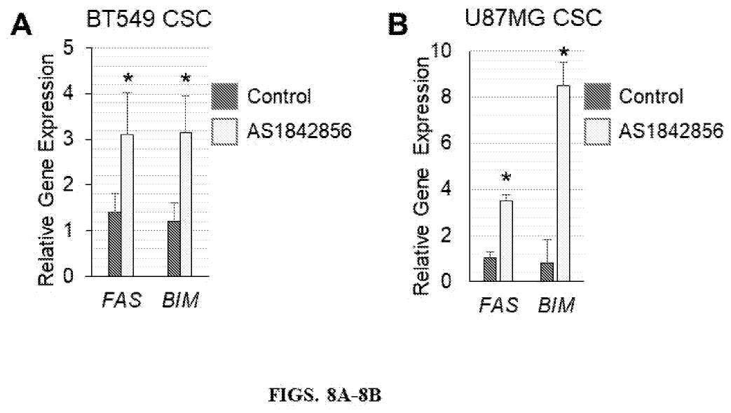

To ascertain the mechanism responsible for reduced cell numbers upon FOXO1 inhibition, qRT-PCR analyses was performed. It was found that selective FOXO1 inhibitor treatment for 48 hours induced FAS (death receptor) and/or BIM (BCL2L11) gene expression in BT549, MDA-MB-468 breast cancer cell lines as well as DBTRG, A172, LN229, LN18 and U87MG GBM cell lines (FIGS. 2A-F and 5A). It was also found that selective FOXO1 inhibitor treatment for 48 hours induced the FAS gene in HCT116 colon cancer cells (FIG. 5B). Treatment of BT549 and MDA-MB-468 cells with another FOXO1 inhibitor AS1708727 for four days led to increased FAS gene expression (FIGS. 5C-5D). Cancer stem cells (CSCs) are often resistant to chemotherapeutic interventions. To determine whether FOXO1 inhibition induced pro-apoptotic genes in this setting, CSCs were isolated from BT549 and U87MG cells. It was found that both that FAS and BIM were induced in CSCs upon AS1842856 treatment (FIGS. 8A-B).

RNAi experiments were performed to ascertain whether reduction of FOXO1 induced apoptotic genes. The siRNA was esiRNA (EHU156591 MISSION® esiRNA; Millipore Sigma) with a CDNA target sequence of SEQ ID NO: 1 (TTCGTGTGCAGAATGAAGGAACTGGAAAAAGTTCTTGGTGGATGCTCAATC CAGAGGGTGGCAAGAGCGGGAAATCTCCTAGGAGAAGAGCTGCATCCATGGAC AACAACAGTAAATTTGCTAAGAGCCGAAGCCGAGCTGCCAAGAAGAAAGCATCT CTCCAGTCTGGCCAGGAGGGTGCTGGGGACAGCCCTGGATCACAGTTTTCCAAAT GGCCTGCAAGCCCTGGCTCTCACAGCAATGATGACTTTGATAACTGGAGTACATT TCGCCCTCGAACTAGCTCAAATGCTAGTACTATTAGTGGGAGACTCTCACCCATT ATGACCGAACAGGATGATCTTGGAGAAGGGGATGTGCATTCTATGGTGTACCCG CCATCTGCCGCAAAGATGGCCTCTACTTTACCCAGTCTGTCTGAGATAAGCAATC CCGAAAACATGGAAA)

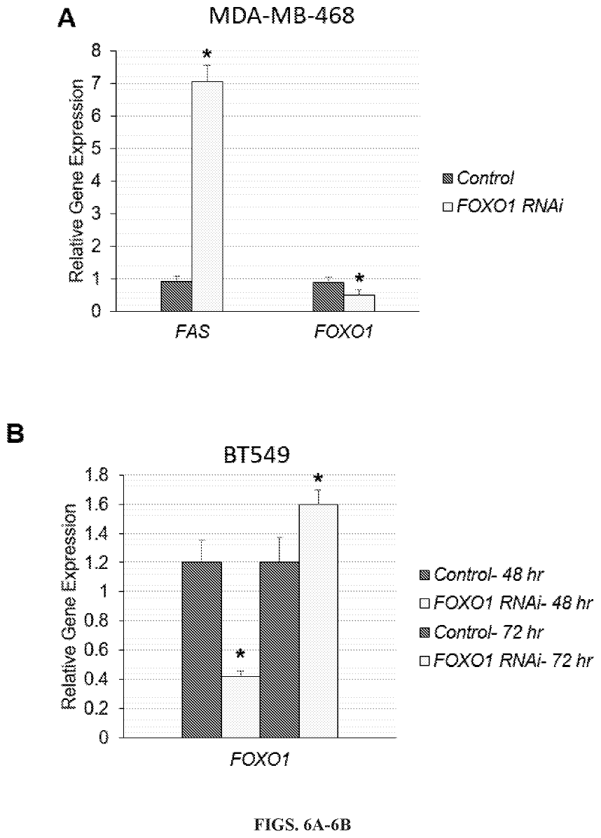

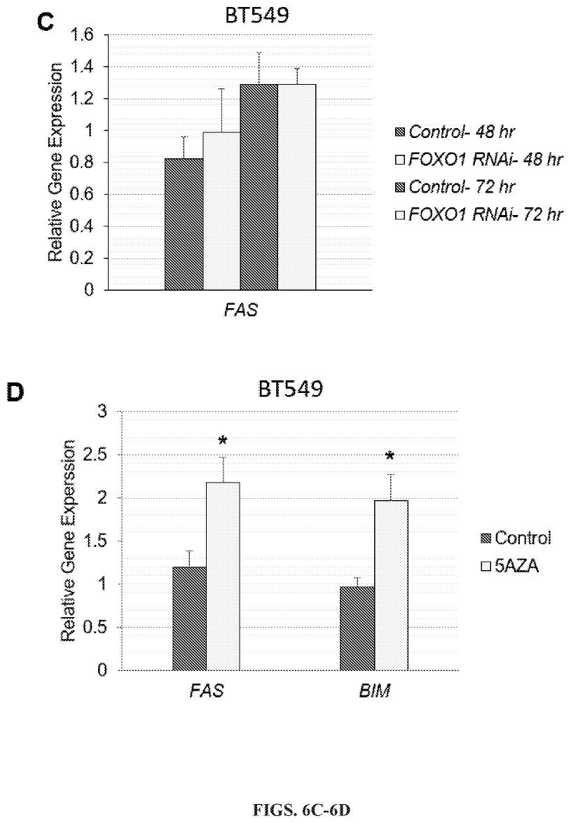

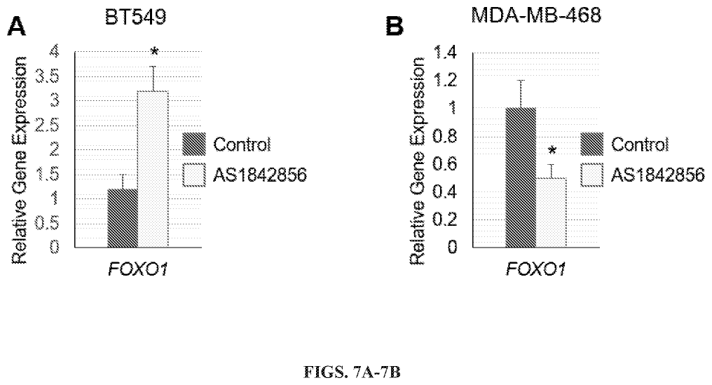

It was found that MDA-MB-468 cells treated with FOXO1 esiRNA had increased FAS gene expression 72 hours post-transfection (FIG. 6A). This supports the notion that reduction of FOXO1 leads to the induction of apoptotic genes. RNAi in BT549 cells was also performed. Forty-eight hours post-transfection samples showed reduced FOXO1 expression but no change in apoptotic gene expression (FIGS. 6B-C). At 72 hours post-transfection, FOXO1 gene expression was increased, whereas FAS gene expression was unchanged (with FOXO1 RNAi in BT549 cells, FIGS. 5B-C). Perhaps, there are feedback mechanisms in BT549 cells that restore FOXO1 expression upon its loss of expression and/or function. In line with this idea, inhibition of FOXO1 with AS1842865 (1 μM for 48 hours) led to increased FOXO1 gene expression in BT549 cells (FIG. 7A). In contrast, decreased FOXO1 expression was observed in MDA-MB-468 cells after 48 hours of AS1842856 treatment (FIG. 7B).

Given that FAS is commonly silenced in cancer to prevent apoptosis, it was examined whether this gene was also silenced in basal breast cancer cells. BT549 cells were treated with 5-AZA to determine whether methylation impacted the expression of apoptotic genes in this context. It was found that 5-AZA treatment (3 μM for 48 hours) induced FAS and BIM in BT549 cells (FIG. 6D). FOXO1 may promote the silencing of apoptotic genes in these cancers.

FOXO1 inhibition led to apoptosis induction based on Caspase 3 cleavage and flow cytometric analyses. To clarify the impact of FOXO1 on BBC and GBM cell viability, BT549 and LN229 cells were treated with AS1842856 for 48 and 72 hours respectively. It was found that AS1842856-treated cells were positive for Annexin V-FITC and/or PI (indicators of apoptosis); see FIGS. 3A-3C. It was also found that AS1842856-treated cells were positive for caspase 3 cleavage by western blot analysis (FIG. 3D).

The role of FOXO1 in cancer and apoptosis is becoming increasingly complex (Hornsveld et al., 2018). Canonically FOXO transcription factors were described as tumor suppressors that induced apoptosis in part by increasing target gene expression, such as TRAIL (Calnan and Brunet, 2008). However, in DLBCL and AML (Acute myeloid leukemia), FOXO factors promote cancer aggressiveness in some instances by sustaining leukemic initiating cells (Trinh et al., 2013). FOXO-1, -3, and -4 are ubiquitously expressed and impact wide-ranging biological processes, including metabolism, cell motility, cell fate, and the cell cycle (Carter and Brunet, 2007).

Inhibition of FOXO1 led to a loss in colony number accompanied by induction of FAS in BBC and GBM cells. This may be due to a loss in cancer stem cells, leading to a loss in signals that prevent apoptosis. FOXO1 sustains AML leukemic initiating cells (Sykes et al., 2011). Loss of FOXO1 led to differentiation and reduced cell numbers (Lin et al., 2017). FOXO1 may be part of (or regulates) the machinery that silences FAS in BBC and GBM. Elegant experiments by Wajapeyee et al. delineated a step-by-step mechanism by which DNMT1 and other factors were recruited to the FAS promoter leading to cytosine methylation (among other things such as methylation of histone H3 on lysine 27) to silence this gene (Wajapayee et al., 2017). Indeed, FAS gene expression was induced by 5-AZA treatment in BT549 cells, suggesting that methylation plays a role in its regulation in this setting (FIG. 6D). Perhaps like its ability to repress CCND1, FOXO1 directly represses FAS in BT549 cells (Dharaneeswaran et al., 2014). Alternatively, FOXO1 may regulate the expression of a required component of the machinery that silences FAS.

Loss of function experiments indicated that at least in part, FOXO1 promotes the viability in a set of BBC and GBM cell lines. It is known that FOXO1 regulates stem genes, but the impact of this function on cellular viability remains to be determined. Researchers have investigated the effects of cancer stem signaling on differentiated glioma cells using U87MG models that harbor oncogene EGFR-VIII (Inda et al., 2010). These cells secrete LIF and IL6, which are required to sustain cancer cell line growth and survival.

While eight of the nine cell lines examined had reduced colony formation upon AS1842856 treatment, U87MG cells were resistant. Therefore, U87MG cells are resistant to AS1842856 treatment even though FOXO1 aids in driving stem genes in this cell line upon NVP-BEZ235 (dual PI3K inhibitor) treatment. Notably, FAS was induced by AS1842856 treatment in U87MG cells (FIG. 3A). Hence, U87MG cells are resistant to apoptotic stimuli driven by a lack of FOXO1. U87MG cells may harbor a mutation that blocks apoptosis induction. U87MG cells are TP53 wildtype (Ianzini et al., 2007). Alternatively, other FOXO factors such as FOXO3 may serve a functionally redundant role with FOXO1 to promote viability in U87MG cells.

Sustained FOXO1 inhibition may be needed for FAS induction. Importantly, FOXO1 RNAi led to robust induction of FAS gene expression in MDA-MB-468 cells (FIG. 6A), corroborating evidence found with FOXO1 inhibitor experiments (FIGS. 2A-F and 5A-B). It was noted that AS1708727 only induced the FAS gene, whereas AS1842856 robustly caused FAS and BIM in the same cell lines BT549 and MDA-MB-468.

Thus, this work highlights FOXO1 as a therapeutic target in poor-prognosis cancers BBC and GBM. Inhibition of FOXO1 by AS1842856 or AS1708727 treatment led to reduced colony formation and apoptotic gene expression. These data reveal novel avenues for therapeutics and insights into the functions of FOXO1 in these cancers.

### Example 2—Materials and Methods

Cell culture and drug treatments. Cell lines were obtained from ATCC (American Type Culture Collection, Manassas, VA) and grown under standard conditions (5% CO₂, 10% FBS (fetal bovine serum), with 5% antifungal/antibacterial). Cell lines were tested for Mycoplasma using the MycoAlert Mycoplasma Detection Kit (Lonza, Basel Switzerland, cat: LT07-218); all experiments were done with mycoplasma negative cells. U87MG cells were propagated in MEM (Minimal Essential Medium). BT549 and DBTRG cells were propagated in RPMI (Roswell Park Memorial Institute 1640 Medium). LN18, U118MG, A172 and LN229 cells were propagated in DMEM (Dulbecco's Modified Eagle Medium). Neurosphere/cancer stem cell cultures for U87MG and BT549 cell lines were plated with 40,000 cells per mL in 3D Tumorsphere Medium XF (Sigma cat: C-28070, Burlington, MA). BT549 cancer stem cell cultures were supplemented with 1XB27 XenoFree CTS (Gibco/Thermo Fisher Waltham, MA, USA). AS1842856 was purchased from Calbiochem (Danvers, MA) and utilized at 200 nM, 500 nM and 1 μM final concentrations as indicated. AS1708727 was purchased from MedChem Express (Monmouth Junction, NJ) and was used at 0.5 μM, 1.0 UM and 2.0 μM concentrations. 5-aza-2′-deoxycytidine (5-AZA) was purchased from Millipore/Sigma (Burlington, MA) and utilized at a final concentration of 3 μM.

Colony formation assays.

Cells were plated at a density of 2,700 cells per mL and were treated five days with indicated drug. Treatments were investigated in triplicate (in numerous independent experiments) and stained with crystal violet. Plates were aspirated of media then each well was washed with 1.0 mL of 1x phosphate buffered saline (PBS) once before being stained with 1.0 mL of crystal violet stain (0.5% crystal violet in buffered formalin) and incubated for 15 minutes. The stain was aspirated, and wells were washed 3 times with 0.5 mL of 1x PBS. After collections were completed, crystal violet-stained plates were solubilized using 0.5 mL on each well of 10% acetic acid and placed on shaker for 1 hour. Solubilized samples were transferred to 96 well plates and quantified on a spectrophotometer at 590 nm. Quantified plates were analyzed with a Tukey test. Error bars were added using the standard deviation.

### Western Blot.

Total protein was obtained from indicated cells by rinsing cells with 1×PBS (phosphate buffered saline) followed by directed lysis in 2× sample buffer (125 mM Tris-HCL at pH 6.8, 2% sodium dodecyl sulfate (SDS), 10% 2-mercaptoethanol, 20% glycerol, 0.05% bromophenol blue, 8 M urea); 2× sample buffer was added to each well and cells scraped with a cell scraper. The lysate was collected from each well, placed into a 1.5 mL microcentrifuge tube and heated for 10 minutes at 95° C. in a dry-bath heat block. Protein lysates were separated by sodium dodecyl sulfate-polyacrylamide gel electrophoresis (SDS-PAGE) at 100V for 1 hour. Resolved proteins were then transferred onto a polyvinylidene fluoride (PVDF) membrane for an hour and 30 minutes then blocked in a 5% milk solution (Carnation powdered milk, 1× Tris-buffered saline with Tween 20 (TBST) for an hour. Membranes were incubated with indicated primary antibody overnight at 4° C. then washed for 20 minutes with TBST in 5-minute intervals. The blot was then incubated with secondary antibody for 1.5 hours. Membranes were washed for 20 minutes in 5-minute intervals and allowed to develop using SuperSignal West Dura Extended Duration Substrate luminol solution (Pierce Biotechnology, Waltham, MA) for 5 minutes. A Bio Rad ChemDoc XRS+Molecular Imager was utilized for protein detection (Bio Rad Hercules, CA). Data was analyzed with NIH Image J. Antibodies were obtained from Cell Signaling Technologies (Danvers, MA): Cleaved Caspase 3 antibody (cat: 94530). Beta-Actin antibody (clone AC-74, cat: A2228) was obtained from Sigma and utilized at a 1:2000 dilution in TBST with 5% non-fat dried milk.

Quantitative real time PCR:

Total RNA was prepared using the Qiagen RNeasy kit (Hilden, Germany), which was then used to generate cDNA using Superscript Reverse Transcriptase II (Invitrogen, Carlsbad, CA). Samples (cDNAs) were analyzed using (Power SYBR Green Master Mix, Applied Biosystems, Foster City, CA) and the ABI Step-One Real-time system (Carlsbad, CA). Expression levels were normalized to Beta-Tubulin, TUBB in gene expression experiments and calculated using 2-4ACT method (Livak et al., 2001). Primer sequences are detailed in Table 1.

TABLE 1
Gene-specific PCR primers. The primers used for gene
expression analysis are detailed.

| Primer | Sequence | SEQ ID NO: |
|---|---|---|
| FAS F (detects FAS cell | 5′ GGGGTGGCTTTGTCTTCTTCTTTTG 3′ | 2 |
| surface death receptor) | | |
| FAS R (detects FAS cell | 5′ ACCTTGGTTTTCCTTTCTGTGCTTTCT 3′ | 3 |
| surface death receptor) | | |
| BIMF (BCL2L11) | 5′ CAAACCCCAAGTCCTCCTT 3′ | 4 |
| BIM R (BCL2L11) | 5′ TCTTGGGCGATCCATATCTC 3′ | 5 |
| TUBB F | 5′ CTGGACCGCATCTCTGTGTA 3′ | 6 |
| TUBB R | 5′ ATCTGGCCAAAAGGACCTG 3′ | 7 |
| FOXO1 F | 5′ ACGAGTGGATGGTCAAGAGC 3′ | 8 |
| FOXO1 R | 5′ CTGCACACGAATGAACTTGC 3′ | 9 |

### RNAi Experiments.

MDA-MB-468 cells were grown to log phase in DMEM with 10% FBS without antibiotics. BT549 cells were grown to log phase in DMEM with 10% FBS without antibiotics. Cells were transfected with FOXO1 esiRNA (EHU156591 Sigma, St. Louis, MO) or EGFP control esiRNA (EHUEGFP) using Lipofectamine 3000 (utilized only L3000 reagent, Invitrogen, Carlsbad, CA).

### Colony Formation Assays.

Cells were plated at 2,700 cells per mL and were treated five days with the indicated drug. Treatments were investigated in triplicate (in numerous independent experiments) and stained with crystal violet. Plates were aspirated of media, then each well was washed with 1.0 mL of 1× phosphate-buffered saline (PBS) once before being stained with 1.0 mL of crystal violet stain (0.5% crystal violet in buffered formalin) and incubated for 15 minutes. The stain was aspirated, and cells were washed three times with 0.5 mL of 1×PBS. After collections were completed, crystal violet-stained plates were solubilized using 0.5 mL on each well of 10% acetic acid and placed on a shaker for 1 hour. Solubilized samples were transferred to 96 well plates and quantified on a spectrophotometer at 590 nm using iMark Microplate Absorbance Reader (Bio-Rad, Hercules, CA). Quantified plates were analyzed with a Tukey Test. Error bars were added using the standard deviation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Anjum et al., Biomed Pharmacother 2017, 92, 681-689.
Ben-Porath et al., Nat Genet 2008, 40 (5), 499-507.
Bertucci et al., Curr Mol Med 2012, 12 (1), 96-110.
Brunet et al., Cell 1999, 96 (6), 857-68.
Brunet et al., J Cell Biol 2002, 156 (5), 817-28.
Calnan and Brunet, Oncogene 2008, 27 (16), 2276-88.
Carter and Brunet, Curr Biol 2007, 17 (4), R113-4.
Chakravarti et al., Int J Radiat Oncol Biol Phys 2004, 58 (3), 927-31.
Dharaneeswaran et al., Circ Res. 2014, 115, 238-251.
Diana et al., Cancers (Basel) 2020, 12 (4).
Firat et al., Oncotarget 2016, 7 (34), 54883-54896.
Hornsveld, et al., Semin Cancer Biol. 2018, 50, 90-100.
Ianzini et al., Radiat Res. 2007, 168, 183-92.
Inda et al., Genes Dev. 2010, 24, 1731-45.
International Patent Publication No. WO 02/016536
International Patent Publication No. WO 03/016496
International Patent Publication No. WO 98/53058
International Patent Publication No. WO 98/53059
International Patent Publication No. WO 98/53060
International Patent Publication No. WO2009/101611
International Patent Publication No. WO2009/114335
International Patent Publication No. WO2015016718
Keniry et al., Genes Dev 2013, 27 (8), 916-27.
Kim et al., Cell Death Differ 2015, 22 (12), 2034-45.
Langlet et al., Cell. 2017 Nov. 2; 171 (4): 824-835.e18.
Lin et al., Blood. 2017, 130, 1213-1222.
Livak et al., Methods 2001, 25 (4), 402-8.
Lu et al., Rev Clin Exp Hematol 2003, 7 (2), 205-28.
Lucas et al., PLOS One 2010, 5 (9).
Martinez et al., J Cancer Res Clin Oncol 2020, 146 (3), 593-604.
Matsumoto and Accili. Cell Metab 2005, 1 (4), 215-6.
Miyamoto et al., Cell Stem Cell 2007, 1 (1), 101-12.
Nagashima et al., Mol Pharmacol 2010, 78 (5), 961-70.
Paik et al., Cell 2007, 128 (2), 309-23.
Paik et al., Cell Stem Cell 2009, 5 (5), 540-53.
Pardoll, Nat Rev Cancer, 12 (4): 252-64, 2012
Pires et al., Cancer Biol Ther 2013, 14 (3), 246-53.
Saal et al., Cancer Res 2005, 65 (7), 2554-9.
Saal et al., Nat Genet 2008, 40 (1), 102-7.
Saal et al., Proc Natl Acad Sci USA 2007, 104 (18), 7564-9.
Salcher et al., Oncogene 39, 1080-1097 (2020).
Sykes et al., Cell. 2011, 146, 697-708.
Tanaka et al., Eur J Pharmacol 2010, 645 (1-3), 185-91.
Trinh et al., Blood 2013, 121 (18), 3666-74.
U.S. Pat. No. 6,140,081
U.S. Pat. No. 6,453,242
U.S. Pat. No. 6,534,261
U.S. Pat. No. 8,008,449
U.S. Pat. No. 8,354,509
U.S. Pat. No. 8,735,553
U.S. Patent Publication No. 2011/0301073
U.S. Patent Publication No. US20110008369
U.S. Patent Publication No. US2014022021
U.S. Patent Publication No. US20140294898
Wajapeyee et al., Genes Dev. 2013, 27, 2221-6.
Zhang et al., Nat Cell Biol 2011, 13 (9), 1092-9.

What is claimed is:

1. A method for treating or preventing cancer in a patient comprising administering an effective amount of a FOXO1 inhibitor to the patient.

2. The method of claim 1, wherein the FOXO1 inhibitor is 5-Amino-7-(cyclohexylamino)-1-ethyl-6-fluoro-4-oxo-1,4-dihydroquinoline-3-carboxylic acid, 2-cyclopentyl-N-[2,4-dichloro-3-(isoquinolin-5-yloxymethyl) phenyl] N-methylacetamide, or 3β-Hydroxy-11-oxoolean-12-en-30-oic acid 3-hemisuccinate.

3. The method of claim 1, wherein the FOXO1 inhibitor comprises short interfering RNA (siRNA), short hairpin (shRNA).

4. The method of claim 3, wherein the FOXO1 inhibitor comprises siRNA.

5. The method of claim 4, wherein the siRNA is endoribonuclease-prepared siRNA (esiRNA).

6. The method of claim 5, wherein the esiRNA comprises a cDNA target sequence of SEQ ID NO: 1.

7. The method of any of claims 1-6, wherein the cancer is an aggressive cancer.

8. The method of any of claims 1-7, wherein the cancer is brain cancer, breast cancer, or colon cancer.

9. The method of claim 8, wherein the brain cancer is glioblastoma.

10. The method of claim 8, wherein the breast cancer is basal breast cancer or triple negative breast cancer.

11. The method any of claims 1-10, wherein the cancer is recurrent.

12. The method of any of claims 1-10, further comprising administering an additional anti-cancer therapy.

13. The method of claim 12, wherein the additional anti-cancer therapy comprises chemotherapy, gene therapy, surgery, hormonal therapy, anti-angiogenic therapy or immunotherapy.

14. The method of claim 12, wherein the additional anti-cancer therapy is chemotherapy.

15. The method of claim 14, wherein the chemotherapy comprises vorinostat, temozolomide, cisplatin, carboplatin, paclitaxel or a combination thereof.

16. The method of claim 12, wherein the additional anti-cancer therapy is a receptor tyrosine kinase inhibitor.

17. The method of claim 16, wherein the receptor tyrosine kinase inhibitor is imatinib.

18. The method of claim 16, wherein the receptor tyrosine kinase inhibitor is an EGFR inhibitor.

19. The method of claim 18, wherein the EGFR inhibitor is erlotinib or gefitinib.

20. The method of claim 12, wherein the additional anti-cancer therapy is trastuzumab.

21. The method of any of claims 12-20, wherein the FOXO1 inhibitor and additional anti-cancer therapy are administered in the same composition.

22. The method of any of claims 12-20, wherein the FOXO1 inhibitor and additional anti-cancer therapy are administered in separate compositions.

23. The method of any of claims 1-22, wherein the patient has cancer cells with increased FOXO1 expression as compared to a control.

24. The method of any of claims 1-23, wherein the method comprises administering more than one additional anti-cancer therapy.

25. The method of any of claims 1-24, wherein the patient has been previously administered an anti-cancer therapy.

26. The method of claim 25, wherein the anti-cancer therapy is chemotherapy.

27. The method of claim 25 or 26, wherein the patient had low or no response to the chemotherapy.

28. The method of any of claims 1-27, wherein the method results in increased apoptotic gene expression as compared to expression prior to administering the FOXO1 inhibitor.

29. The method of claim 28, wherein the increased apoptotic gene expression comprises increased expression of FAS and BIM.

30. The method of any of claims 1-29, wherein the patient is a human.

31. The method of any of claims 1-30, wherein the FOXO1 inhibitor and/or additional anti-cancer therapy are administered two or more times.

32. A composition comprising an effective amount of a FOXO1 inhibitor for use in the treatment or prevention of a cancer in a patient.

33. The composition of claim 32, wherein the FOXO1 inhibitor is 5-Amino-7-(cyclohexylamino)-1-ethyl-6-fluoro-4-oxo-1,4-dihydroquinoline-3-carboxylic acid, 2-cyclopentyl-N-[2,4-dichloro-3-(isoquinolin-5-yloxymethyl) phenyl] N-methylacetamide, or 3β-Hydroxy-11-oxoolean-12-en-30-oic acid 3-hemisuccinate.

34. The composition of claim 32, wherein the FOXO1 inhibitor comprises siRNA or shRNA.

35. The composition of claim 32, wherein the composition further comprises an additional anti-cancer therapy.

36. The composition of claim 35, wherein the additional anti-cancer therapy comprises chemotherapy, gene therapy, surgery, hormonal therapy, anti-angiogenic therapy or immunotherapy.

37. The composition of claim 36, wherein the additional anti-cancer therapy is chemotherapy.

38. The composition of claim 37, wherein the chemotherapy comprises vorinostat, temozolomide, cisplatin, carboplatin, paclitaxel or a combination thereof.

39. The composition of claim 36, wherein the additional anti-cancer therapy is a receptor tyrosine kinase inhibitor.

40. The composition of claim 39, wherein the receptor tyrosine kinase inhibitor is imatinib.

41. The composition of claim 39, wherein the receptor tyrosine kinase inhibitor is an EGFR inhibitor.

42. The composition of claim 41, wherein the EGFR inhibitor is erlotinib or gefitinib.

43. The composition of claim 36, wherein the additional anti-cancer therapy is trastuzumab.

44. Use of a composition comprising an effective amount of a FOXO1 inhibitor for the treatment or prevention of cancer in a patient.

45. The use of claim 44, wherein the FOXO1 inhibitor is 5-Amino-7-(cyclohexylamino)-1-ethyl-6-fluoro-4-oxo-1,4-dihydroquinoline-3-carboxylic acid, 2-cyclopentyl-N-[2,4-dichloro-3-(isoquinolin-5-yloxymethyl) phenyl] N-methylacetamide, or 3β-Hydroxy-11-oxoolean-12-en-30-oic acid 3-hemisuccinate.

46. The use of claim 44, wherein the FOXO1 inhibitor comprises siRNA or shRNA.

47. The use of any of claims 44-46, wherein the use further comprises an additional anti-cancer therapy.

48. The use of claim 47, wherein the additional anti-cancer therapy comprises chemotherapy, gene therapy, surgery, hormonal therapy, anti-angiogenic therapy or immunotherapy.

49. The use of claim 47, wherein the additional anti-cancer therapy is chemotherapy.

50. The use of claim 49, wherein the chemotherapy comprises vorinostat, temozolomide, cisplatin, carboplatin, paclitaxel or a combination thereof.

51. The use of claim 47, wherein the additional anti-cancer therapy is a receptor tyrosine kinase inhibitor.

52. The use of claim 51, wherein the receptor tyrosine kinase inhibitor is imatinib.

53. The use of claim 51, wherein the receptor tyrosine kinase inhibitor is an EGFR inhibitor.

54. The use of claim 53, wherein the EGFR inhibitor is erlotinib or gefitinib.

55. The use of claim 47, wherein the additional anti-cancer therapy is trastuzumab.


SEQUENCE LISTING (9 sequences)

SEQ ID NO: 1 - DNA, 447 bp, synthetic construct
  source 1..447: mol_type = other DNA
ttcgtgtgcagaatgaaggaactggaaaaagttcttggtggatgctcaatccagagggtggcaagagcgggaaatctcctaggagaagagctgcatccatggacaacaacagtaaatttgctaagagccgaagccgagctgccaagaagaaagcatctctccagtctggccaggagggtgctggggacagccctggatcacagttttccaaatggcctgcaagccctggctctcacagcaatgatgactttgataactggagtacatttcgccctcgaactagctcaaatgctagtactattagtgggagactctcacccattatgaccgaacaggatgatcttggagaaggggatgtgcattctatggtgtacccgccatctgccgcaaagatggcctctactttacccagtctgtctgagataagcaatcccgaaaacatggaaa

SEQ ID NO: 2 - DNA, 25 bp, synthetic construct
  source 1..25: mol_type = other DNA
ggggtggctttgtcttcttcttttg

SEQ ID NO: 3 - DNA, 27 bp, synthetic construct
  source 1..27: mol_type = other DNA
accttggttttcctttctgtgctttct

SEQ ID NO: 4 - DNA, 19 bp, synthetic construct
  source 1..19: mol_type = other DNA
caaaccccaagtcctcctt

SEQ ID NO: 5 - DNA, 20 bp, synthetic construct
  source 1..20: mol_type = other DNA
tcttgggcgatccatatctc

SEQ ID NO: 6 - DNA, 20 bp, synthetic construct
  source 1..20: mol_type = other DNA
ctggaccgcatctctgtgta

SEQ ID NO: 7 - DNA, 19 bp, synthetic construct
  source 1..19: mol_type = other DNA
atctggccaaaaggacctg

SEQ ID NO: 8 - DNA, 20 bp, synthetic construct
  source 1..20: mol_type = other DNA
acgagtggatggtcaagagc

SEQ ID NO: 9 - DNA, 20 bp, synthetic construct
  source 1..20: mol_type = other DNA
ctgcacacgaatgaacttgc